SEQUENCE LISTING (45 sequences)

SEQ ID NO: 1 - protein, 23 aa, Artificial Sequence
  peptide domain
DPAALKRARXTEXXRRXRARKLQ

SEQ ID NO: 2 - protein, 52 aa, Artificial Sequence
  leucine-zipper fusion protein
DPAALKRARXTEXXRRXRARKLQLEDKVEELLSKNYHLENEVARLKKLVGER

SEQ ID NO: 3 - DNA, 69 bp, Artificial Sequence
  peptide domain-encoding nucleic acid
gatcctgctgctctaaaacgtgctaganncactgaannnnnnaggcgtnnncgtgcgagaaagttgcaa

SEQ ID NO: 4 - DNA, 156 bp, Artificial Sequence
  leucine-zipper fusion protein-encoding nucleic acid
gatcctgctgctctaaaacgtgctaganncactgaannnnnnaggcgtnnncgtgcgagaaagttgcaacttgaagacaaggttgaagaattgctttcgaaaaattatcacttggaaaatgaggttgccagattaaagaaattagttggcgaacgc

SEQ ID NO: 5 - DNA, 586 bp, Artificial Sequence
  coding nucleic acid with nuclear transport sequence and transcription activating sequence
atggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatcgaattcgatcctgctgctctaaaacgtgctagannccactgaannnnnnaggcgtnnncgtgcgagaaagttgcaacttgaagacaaggttgaagaattgctttcgaaaaattatcacttggaaaatgaggttgccagattaaagaaattagttggcgaacgctgaggatcccca

SEQ ID NO: 6 - protein, 191 aa, Artificial Sequence
  peptides containing nuclear transport sequence and transcription-activating sequence
MDKAELIPEPPKKKRKVELGTAANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKEIEFDPAALKRARXTEXXRRXRARKLQLEDKVEELLSKNYHLENEVARLKKLVGER

SEQ ID NO: 7 - DNA, 49 bp, Artificial Sequence
  3xE2Fwt sense sequence
aaagcgcgcgaaactaaagcgcgcgaaactaaagcgcgcgaaactagct

SEQ ID NO: 8 - DNA, 49 bp, Artificial Sequence
  3xE2Fwt antisense sequence
agtttcgcgcgctttgatttcgcgcgctttgatttcgcgcgctttgatc

SEQ ID NO: 9 - DNA, 6838 bp, Artificial Sequence
  plasmid pH/Si3xE2Fwt
gaattcccgggaggtacaaagcgcgcgaaactaaagcgcgcgaaactaaagcgcgcgaaactagctcttacgcgttcgcgaatcgatccgcggtctagaaattcctggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtccccgaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggtttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccctagagtcgacctgcaggcatgcaagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggc

SEQ ID NO: 10 - DNA, 175 bp, Artificial Sequence
  peptide domain-encoding nucleic acid
gggaattcgatcctgctgctctaaaacgtgctaganncactgaannnnnnaggcgtnnncgtgcgagaaagttgcaacttgaagacaaggttgaagaattgctttcgaaaaattatcacttggaaaatgaggttgccagattaaagaaattagttggcgaacgctgaggatcccc

SEQ ID NO: 11 - DNA, 175 bp, Artificial Sequence; identical to SEQ ID NO: 10
  vector pGADD424

SEQ ID NO: 12 - DNA, 22 bp, Artificial Sequence
  primer
gatgtatataactatctattcg

SEQ ID NO: 13 - protein, 20 aa, Artificial Sequence
  consensus sequence
DPAALKRARGTEVVRRGRAR

SEQ ID NO: 14 - protein, 20 aa, Artificial Sequence
  3xE2Fwt sequence-recognizing domain
DPAALKRARCTEVMRRQRAR

SEQ ID NO: 15 - protein, 20 aa, Artificial Sequence
  3xE2Fwt sequence-recognizing domain
DPAALKRARPTENVRRGRAR

SEQ ID NO: 16 - protein, 20 aa, Artificial Sequence
  3xE2Fwt sequence-recognizing domain
DPAALKRARNTEVTRRSRAR

SEQ ID NO: 17 - protein, 20 aa, Artificial Sequence
  3xE2Fwt sequence-recognizing domain
DPAALKRARVTENSRRDRAR

SEQ ID NO: 18 - protein, 20 aa, Artificial Sequence
  3xE2Fwt sequence-recognizing domain
DPAALKRARHTETSRRIRAR

SEQ ID NO: 19 - protein, 20 aa, Artificial Sequence
  3xE2Fwt sequence-recognizing domain
DPAALKRARVTEVIRRGRAR

SEQ ID NO: 20 - protein, 20 aa, Artificial Sequence
  3xE2Fwt sequence-recognizing domain
DPAALKRARITEGIRRLRAR

SEQ ID NO: 21 - protein, 20 aa, Artificial Sequence
  3xE2Fwt sequence-recognizing domain
DPAALKRARSTELNRRGRAR

SEQ ID NO: 22 - protein, 20 aa, Artificial Sequence
  3xE2Fwt sequence-recognizing domain
DPAALKRARGTERLRRGRAR

SEQ ID NO: 23 - protein, 20 aa, Artificial Sequence
  3xE2Fwt sequence-recognizing domain
DPAALKRARGTEATRRVRAR

SEQ ID NO: 24 - protein, 20 aa, Artificial Sequence
  3xE2Fwt sequence-recognizing domain
DPAALKRARCTEEVRRWRAR

SEQ ID NO: 25 - protein, 20 aa, Artificial Sequence
  3xE2Fwt sequence-recognizing domain
DPAALKRARCTEVQRRGRAR

SEQ ID NO: 26 - protein, 20 aa, Artificial Sequence
  3xE2Fwt sequence-recognizing domain
DPAALKRARDTEMLRRCRAR

SEQ ID NO: 27 - protein, 20 aa, Artificial Sequence
  3xE2Fwt sequence-recognizing domain
DPAALKRARDTEMVRRARAR

SEQ ID NO: 28 - protein, 20 aa, Artificial Sequence
  3xE2Fwt sequence-recognizing domain
DPAALKRARGTEVVRRCRAR

SEQ ID NO: 29 - protein, 20 aa, Artificial Sequence
  3xE2Fwt sequence-recognizing domain
DPAALKRARGTEVVRRCRAR

SEQ ID NO: 30 - protein, 20 aa, Artificial Sequence
  3xE2Fwt sequence-recognizing domain
DPAALKRARITENARRGRAR

SEQ ID NO: 31 - protein, 20 aa, Artificial Sequence
  3xE2Fwt sequence-recognizing domain
DPAALKRARCTEEMRRGRAR

SEQ ID NO: 32 - protein, 20 aa, Artificial Sequence
  3xE2Fwt sequence-recognizing domain
DPAALKRARCTEPSRRGRAR

SEQ ID NO: 33 - protein, 20 aa, Artificial Sequence
  3xE2Fwt sequence-recognizing domain
DPAALKRARNTESGRRTRAR

SEQ ID NO: 34 - protein, 20 aa, Artificial Sequence
  3xE2Fwt sequence-recognizing domain
DPAALKRARDTEGDRRRRAR

SEQ ID NO: 35 - protein, 20 aa, Artificial Sequence
  3xE2Fwt sequence-recognizing domain
DPAALKRARGTEAERRGRAR

SEQ ID NO: 36 - protein, 20 aa, Artificial Sequence
  3xE2Fwt sequence-recognizing domain
DPAALKRARRTELLRRHRAR

SEQ ID NO: 37 - protein, 20 aa, Artificial Sequence
  3xE2Fwt sequence-recognizing domain
DPAALKRARITENARRGRAR

SEQ ID NO: 38 - protein, 20 aa, Artificial Sequence
  3xE2Fwt sequence-recognizing domain
DPAALKRARITEMGRRKRAR

SEQ ID NO: 39 - protein, 20 aa, Artificial Sequence
  3xE2Fwt sequence-recognizing domain
DPAALKRARCTEYCRRIRAR

SEQ ID NO: 40 - protein, 20 aa, Artificial Sequence
  3xE2Fwt sequence-recognizing domain
DPAALKRARGTEEYRRHRAR

SEQ ID NO: 41 - protein, 20 aa, Artificial Sequence
  3xE2Fwt sequence-recognizing domain
DPAALKRARSTELTRRIRAR

SEQ ID NO: 42 - protein, 20 aa, Artificial Sequence
  3xE2Fwt sequence-recognizing domain
DPAALKRARGTEPVRRSRAR

SEQ ID NO: 43 - protein, 20 aa, Artificial Sequence
  3xE2Fwt sequence-recognizing domain
DPAALKRARGTEAERRGRAR

SEQ ID NO: 44 - protein, 20 aa, Artificial Sequence
  3xE2Fwt sequence-recognizing domain
DPAALKRARDTEASRRMRAR

SEQ ID NO: 45 - protein, 20 aa, Artificial Sequence
  3xE2Fwt sequence-recognizing domain
DPAALKRARNTESGRRTRAR

(19) United States
(12) Patent Application Publication
(10) Pub. No.: US 2004/0072180 A1
(43) Pub. Date: Apr. 15, 2004

(54) Dna binding peptide domains and a method for providing domains of this type

(76) Inventors: Andreas Kappel, Konigstein (DE); Norbert Windhab, Hofheim (DE); Thomas Wagner, Arlington, VA (US); Stefan Kienle, Frankfurt (DE); Karsten Kuhn, Dortmund (DE)

Correspondence Address: CLARK & ELBING LLP, 101 FEDERAL STREET, BOSTON, MA, 02110, US

(21) Appl. No.: 10/344,620

(22) PCT Filed: Aug. 20, 2001
(86) PCT No.: PCT/EP01/09565

(30) Foreign Application Priority Data
Aug. 22, 2000 (DE) 100 41 126.6

(51) Int. Cl. C12Q 1/68; C07H 21/04; C12N 9/64; C12P 21/02; C12N 5/06

(52) U.S. Cl. 435/6; 435/69.1; 435/226; 435/320.1; 435/325; 536/23.2

(57) ABSTRACT

The present invention relates to peptidic domains which can be readily synthesized and which can specifically recognize and bind nucleic acid sequences and to a method for finding and providing peptide domains which bind specifically to DNA and biomorphic factors derived therefrom, in particular transcription factors and repressors.

The present invention relates to peptidic domains which can be readily synthesized and which can specifically recognize and bind nucleic acid sequences and to a method for finding and providing peptide domains which bind specifically to DNA and biomorphic factors derived therefrom, in particular transcription factors and repressors.

The transcription of DNA into RNA is the first step of gene expression. The strength of transcription of a gene and thus the amount of RNA formed is determined by regulatory promotor elements and enhancer elements which are associated with the genes and to which transcription factors bind. These proteins bind, by means of a DNA-binding domain, sequence-specifically to short DNA sections in the regulatory elements of said genes. Apart from the DNA-binding domain, transcription factors comprise one or more activator or repressor domains. Activator domains increase transcription of the gene corresponding to the regulatory element bound by the transcription factor, while repressor domains reduce transcription of said gene. Both actions are carried out by the activator or repressor domains recruiting further proteins via protein-protein interactions. The DNA sequence of the regulatory elements of a gene and the protein domains specifically binding to this sequence therefore act as complementary addresses which together locate activator and repressor domains which, in the end, determine the strength of transcription of a gene.

Since many diseases, such as, for example, cancer are caused by deregulated expression of particular genes, various methods have been developed by which transcription of said genes can be influenced and thus converted from a pathological into a physiological state. Apart from substances which inhibit later stages of gene expression, such as, for example, antisense RNA or ribozymes, methods and substances have also been developed which interfere with the earlier stages of gene expression, i.e. binding of transcription factors and transcription. Thus, for example, substances from the substance class of polyamides have been developed, which can bind to regulatory elements of selected genes. Polyamides bind to the minor groove of DNA and thus prevent binding of the most activating transcription factors to said regulatory elements (J. M. Gottesfeld et al., Nature, 1997, 387, 202-205). As a result, polyamides can indirectly reduce transcription of the particular gene. However, DNA binding of polyamide is not very specific so that these substances are not suitable for therapeutic use. In addition, it is not possible to increase the rate of transcription by using polyamides.

In a further method, relatively large amounts of short double-stranded DNA molecules are introduced into cells, which molecules contain a binding site for a transcription factor which is responsible for deregulated transcription of the gene in question. In this “decoy” strategy, the selected transcription factor is competitively blocked and can thus no longer bind to the binding sites in the regulatory elements of its target genes, resulting in the indirect inhibition of transcription of said genes (overview in Suda et al., Endocr. Rev., 1999, 20, 345-357; S. Yla-Hertttuala and J. F. Martin, The Lancet 355, 213-222, 2000).

In recent years, synthetic proteins and peptides have been developed which bind DNA by means of other structural motifs such as, for example, the helix-turn-helix motif, the basic leucine zipper or the α-helix motif. These peptides and proteins are derived from naturally occurring transcription factors and retain the DNA-binding specificity of the particular factor. These peptides usually have a very low affinity for DNA and cannot bind therefore to the particular DNA element under physiological conditions. In some cases, however, binding is increased, compared with the unmodified protein or peptide, by mutagenesis and chemical modification (Z. Shang et al., Proc. Natl. Acad. Sci. USA, 1994, 91, 8373-8377). However, such artificially optimized DNA-binding proteins are suited only to influence regulation of those genes which also respond to the transcription factor from which said proteins have been derived.

A relatively new method for regulating transcription are synthetic transcription factors from the class of zinc finger proteins of the Cys₂-His₂ [lacuna] (D. J. Segall, B. Dreier, R. R. Beerli, C, F. Barbas III, Proc. Natl. Acad. Sci. USA, 1999, 96, 2758-2763). These factors bind to the major groove of DNA by means of a finger-like structure in which a short α-helix and an antiparallel β-sheet are complexed via a zinc atom (D. Rhodes, A. Klug, Sci. Am., 1993, 268, 32-39). The DNA-binding activity of the zinc finger proteins is composed in a modular manner. Individual fingers which bind, in each case, specifically to a different trinucleotide sequence (usually 5′-GNN-3′, where N can be any of the 4 nucleotides) can be combined with one another in a larger polypeptide so that also relatively long DNA sequences which are unique for the target gene to be regulated can be specifically bound (Q. Liu, D. J. Segal, J. B. Ghiara, C. F. Barbas III, Proc. Natl. Acad. Sci. USA, 1997, 94, 5525-5530; R. R. Beerli, D. J. Segal, B. Dreier, C. F. Barbas III, Proc. Natl. Acad. Sci. USA, 1998, 95, 14628-14633). These multifinger proteins are usually encoded in a phage library, and it is therefore possible to isolate those members of the library which bind to a specific DNA sequence of the target gene by means of the phage display method. The cDNA of the DNA-binding polypeptide is subsequently fused to the cDNA of an activator or repressor domain; the resulting fusion protein is thus a specific regulator of the target gene (method overview in D. J. Segal and C. F. Barbas III, Curr. Opin Chem. Biol., 2000, 4, 34-39; A. Klug, J. Mol. Biol., 1999, 293, 215-218). Recently, it has been possible to show, for the first time, that these fusion proteins can in principle regulate transcription of chromosomally located genes (R. R. Beerli, B. Dreier, C. F. Barbas, Proc. Natl. Acad. Sci. USA, 2000, 97, 1495-1500). However, the fact that relatively large proteins such as zinc finger proteins can be transported through cell membranes only with difficulty and frequently lose their original conformation in the process, is a disadvantage. In contrast to polyamides and oligonucleotides, it is, in addition, hardly possible to synthesize C₂H₂ zinc finger proteins, due to their size, so that the proteins can be generated only in relatively small amounts by means of overexpression in microorganisms. Furthermore, finding specifically binding artificial zinc finger proteins is very complicated, since large protein banks need to be screened, owing to the potentially large variety of protein structures.

Against this background, it is the object to provide short peptidic biomorphic factors which can specifically recognize and bind nucleic acid sequences and to provide a method for finding such biomorphic factors.

The object is achieved by peptides comprising the following amino acid sequence or a structural variant thereof:

DPAALKRARXTEXXRRXRARKLQ (Seq. ID No. 1)

Preferably, any two peptides which form, via a suitable linker, a homo- or heterodimeric molecule or a mixture of homo- and heterodimeric molecules are in each case selected from this group.

Structural variants mean amino acid sequences which are at least 75% homologous, preferably more than 90% homologous, to Seq. ID No. 1. Preference is given to functional variants which can bind to DNA in a sequence-specific manner.

Suitable linkers are any compounds which ensure dimerization of two peptides from the group having Seq. ID No. 1 or their structural variants in solution. The linkers may be free organic or inorganic substances which can be added to the peptides of the invention. Examples thereof are, for example, complexing agents which can form coordinative bonds with the free amino or carboxy termini of peptides. In addition, the linkers and peptides may be linked covalently due to chemical modification of at least one of said peptides. Peptidic linkers may also be fused directly to at least one of said peptides.

Preference is given to fusion proteins which have a leucine-zipper amino acid domain in addition to a DNA-binding domain according to Seq. ID No. 1 or to a structural variant. Seq. ID No. 2 represents by way of example the amino acid sequence of a fusion protein of this kind.

DPAALKRARXTEXXRRXRARKLQ LED KVE E LLSKNYHLEN EVARLKKLVGE R Seq. ID No. 2

The peptides of the invention may also be synthesized via methods known to the skilled worker (e.g. by Merryfield synthesis), the peptides can also be obtained using molecular biological methods such as expression in cell culture or in fermenters.

Surprisingly, it has been found that the short dimers which contain peptides with Seq. ID No. 1 or variants thereof specifically recognize DNA sequences and thus can regulate the activity of genes in living cells. Due to the short length of the amino acid sequence, the peptides can be readily synthesized and, in addition, transport through cell membranes is facilitated owing to the short length of said peptide. The high conformational stability of the peptides of the invention ensures that their function, i.e. the ability to bind DNA sequence-specifically, is retained during transport through cell membranes. Moreover, the different structure of the peptides compared with other artificial transcription factors enables better binding of particular DNA sequence motifs.

For these reasons, the dimers of the invention are a suitable module for constructing artificial activating or repressing transcription factors. For this purpose, the DNA-binding domains are fused to known nuclear transport signal domains, transcription-activating domains or transcription-inhibiting domains, such as, for example, the nuclear localization signal of SV40 T-antigen, the transcription activator domain of the viral VP16 protein or the KRAB repressor domain. The artificial biomorphic factors generated in this way can be used for regulating transcription and thus also for regulating gene expression. Thus, the artificial biomorphic factors of the invention make it possible to study phenotypical effects of the change in expression of individual genes in vivo. Especially the use of such biomorphic factors for controling diseases which are based on misregulated transcription of genes, such as, for example, cancer, inflammatory reactions or predictive disorders, is of great interest. The use of said biomorphic factors as biopharmaceuticals has the further advantage of easy physiological degradability of said factors.

The present invention therefore also relates to a pharmaceutical which contains the peptide monomers or dimers of the invention and, where appropriate, suitable additives or excipients and to a method for producing a pharmaceutical for the treatment of disorders based on misregulated expression or on expression of mutated genes, in which method a peptide monomer or dimer of the invention is formulated with pharmaceutically acceptable additives and/or excipients.

The invention further relates to the DNA sequences (Seq. ID No. 3) encoding the inventive peptides according to Seq. ID No. 1 or structural variants thereof or to nucleic acids comprising these sequences.

(Seq. ID No. 3)

GATCCTGCTGCTCTAAAACGTGCTAGANNCACTGAANNNNNNAGGCGTNN
NCGTGCGAGAAAGTTGCAA

Structural variant means nucleic acids with deviating sequence which encode proteins according to Seq. ID No. 1 or structural variants thereof. Preference is given to nucleic acids which encode functional variants of said proteins.

Preference is given to DNA sequences which code for fusion proteins composed of the DNA-binding domains of the invention and of a leucine-zipper domain (Seq. ID No. 4).

(Seq. ID No. 4)

GATCCTGCTGCTCTAAAACGTGCTAGANNCACTGAANNNNNNAGGCGTNN
NCGTGCGAGAAAGTTGCAACTTGAAGACAAGGTTGAAGAATTGCTTTCGA
AAAATTATCACTTGGAAAATGAGGTTGCCAGATTAAAGAAATTAGTTGGC
GAACGC

In addition, the nucleic acids of the invention may contain further coding regions, for example for nuclear transport signal domains (nuclear localization signals), transcription-activating domains (e.g. from transcription factors) or transcription-inhibiting domains.

The nucleic acids of the invention can, for example, be chemically synthesized, for example according to the phosphotriester method (see, for example, Uhlman, E. & Peyman, A. (1990) Chemical Reviews, 90, 543, No. 4), on the basis of the sequences disclosed in Seq. ID No. 3 or 4 or on the basis of the peptide sequences disclosed in SEQ. ID No. 1 or 2 or structural variants thereof by utilizing the genetic code.

The deoxyribonucleic acids of the invention may be used for the purposes of gene therapy or else for studying phenotypical effects due to the change in expression of individual genes in vivo. For this purpose, DNA fragments must have been introduced into suitable expression vectors.

Vectors which may be used are any suitable prokaryotic or eukaryotic expression vectors, depending on the organism used for expressing. Preference is given to commercially available expression vectors which [lacuna] regulatory sequences suitable for the host cell, such as, for example, the trp promotor for expression in E. coli or the ADH-2 promotor for expression in yeast, the baculovirus polyhedrin promotor for expression in insect cells.

Examples of vectors effective in gene therapy are virus vectors, preferably adenovirus vectors, in particular replication-efficient adenovirus vectors, or adeno-associated virus vectors, for example an adeno-associated virus vector which consists exclusively of two inserted terminal repeats (ITR).

Suitable adenovirus vectors are described, for example, in McGrory, W. J. et al. (1988) Virol. 163, 614; Gluzman, Y. et al. (1982) in “Eukaryotic Viral Vectors”(Gluzman, Y. ed.) 187, Cold Spring Harbor Press, Cold Spring Harbor, N.Y.; Chroboczek, J. et al. (1992) Virol. 186, 280; Karlsson, S. et al. (1986) EMBO J. 5, 2377 or WO95/00655.

Suitable adeno-associated virus vectors are described, for example, in Muzyczka, N. (1992) Curr. Top. Microbiol. Immunol. 158, 97; WO95/23867; Samulski, R. J. (1989) J. Virol, 63, 3822; WO95/23867; Chiorini, J. A. et al. (1995) Human Gene Therapy 6, 1531 or Kotin, R. M. (1994) Human Gene Therapy 5, 793.

Vectors effective in gene therapy can also be obtained by complexing the nucleic acid of the invention with liposomes. For this purpose, lipid mixtures as described in Felgner, P. L. et al. (1987) Proc. Natl. Acad. Sci, USA 84, 7413; Behr, J. P. et al. (1989) Proc. Natl. Acad. Sci. USA 86, 6982; Felgner, J. H. et al. (1994) J. Biol. Chem. 269, 2550 or Gao, X. & Huang, L. (1991) Biochim. Biophys. Acta 1189, 195 can be used. The liposomes are prepared by binding the DNA ionically to the surface of said liposomes, mainly in a ratio so as for a positive net charge to remain and for said DNA to be completely complexed by said liposomes.

In a further embodiment, the nucleic acids of the invention are therefore in an expression vector, preferably in an expression vector suitable for the use in gene therapy or for the production of transgenic microorganisms, plants or animals.

Therefore, the present invention further relates to a pharmaceutical which is suitable for application in gene therapy of disorders which are based on misregulated expression or on expression of mutated genes by using the nucleic acids of the invention, where appropriate using further additives or excipients. Especially suitable is a pharmaceutical which contains the inventive nucleic acids in naked form or in the form of one of the above-described vectors effective in gene therapy, or complexed with liposomes.

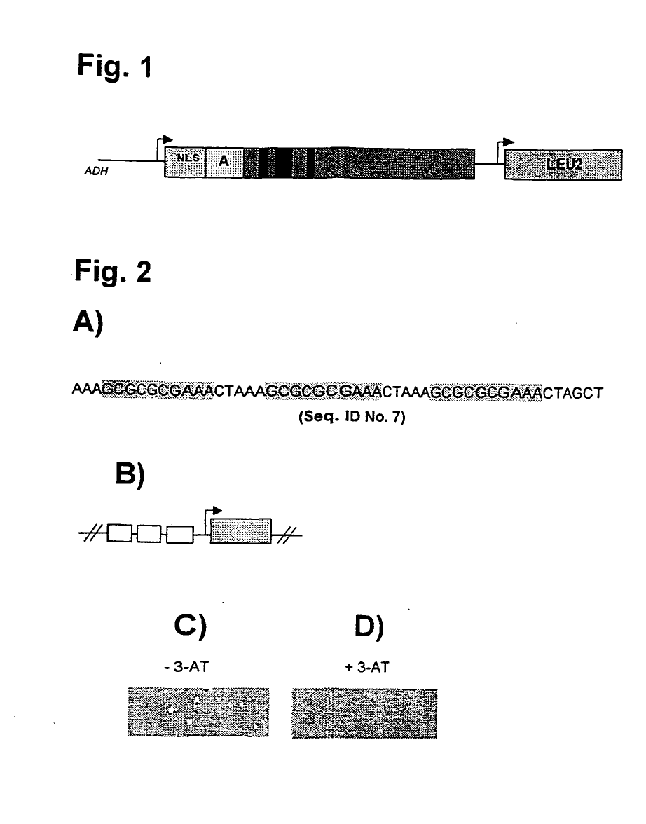

Examples of suitable additives and/or excipients are a physiological salt solution, stabilizers, protease inhibitors, nuclease inhibitors, etc. The invention further relates to a library comprising deoxyribonucleic acids which have the sequences according to Seq. ID No. 3 or a structural variant thereof and whose coding regions contain a transcription-activating domain (AD) and, where appropriate, a nuclear transport sequence domain (NLS) in an open reading frame. In addition, preference is given to said DNAs additionally containing a linker-encoding, particularly preferably a leucine zipper-encoding region (FIG. 1).

The peptide monomer-encoding components of the library form in solution, preferably under physiological conditions, homo- and/or heterodimeric biomorphic transcription factors.

Seq. ID No. 5 and FIG. 1 depict, by way of example, the structural composition of the members of a library containing deoxyribonucleic acids comprising NLS- and AD-encoding regions.

In a preferred embodiment, the library contains DNA sequences which code for individual monomers of the biomorphic transcription factors and whose peptide monomers, after expression in a transformed expression system such as, for example, in E. coli or in yeasts, dimerize via suitable linkers.

Suitable vectors which may be used are, depending on the expression system, any prokaryotic and/or eukaryotic expression vectors. Preference is given to commercially available expression vectors which have a constitutive or inducible promotor, such as, for example, the pGADD424 vector from Clontech Laboratories GmbH, Tullastrasse 4, 69126 Heidelberg, Germany. Generally, the expression vectors also contain regulatory sequences suitable for the host cell, such as, for example, the trp promotor for expression in E. coli or the ADH-2 promotor for expression in yeasts, the baculovirus polyhedrin promotor for expression in insect cells.

Preference is given to libraries which contain one or several copies of all possible cDNA sequences according to Seq. ID No. 3 or to a structural variant. The DNA sequences of the library may be present and stored in sequential form, in the form of a suitable vector or in the form of a cellular expression system transformed with said vectors.

The invention furthermore comprises the biomorphic transcription factors which are expressible with the aid of said libraries. In this way, it is also possible to obtain pure peptide libraries of biomorphic transcription factors. It is also possible to carry out artificial synthesis of biomorphic transcription factors to construct a peptide library. Seq. ID No. 6 depicts, by way of example, the structural composition of the members of a library comprising peptides.

A further subject matter is a method for finding peptidic biomorphic factors which can specifically recognize and bind nucleic acid sequences.

For this purpose, a cellular expression system is chosen in which an essential gene is defective. The cellular expression system is transformed with a corresponding wild-type gene that is controled by a promotor permitting a basal transcription (insertion element). Secondly, the transforming construct contains a DNA sequence which is to be assayed for sequence-specifically binding biomorphic factors (response element).

Suitable response elements are especially regulatory DNA sequences such as promotors or operators. However, gene-specific DNA sequences of the coding region of a gene, in particular specific DNA sequences of a mutated region of a gene, are also preferred subjects to be tested.

Suitable cellular expression systems are any lethal defective mutants which can be cultured via inhibitable basal transcription of an introduced wild-type gene or via a gene product which can be inhibited in a direct and lethal manner. Preference is given to using readily culturable microorganisms such as, for example, E. coli or yeasts, but cell cultures of higher organisms may also be used as expression systems.

It is possible to use, by way of example, known HIS⁻ yeast cells as an expression system. The yeast cells are transformed with a construct that comprises a DNA sequence containing one or multiple copies of a wild type-binding site, for example for transcription factors, enhancers or repressors. The double-stranded sequences to be assayed for binding, biomorphic factors may be inserted into a vector containing a HIS-gene wild type, such as the pHISi (Clontech, Heidelberg, Germany). The HIS gene of the pHISi vector is under the control of a minimal promotor.

The inventive method for finding biomorphic transcription factors which bind DNA in a sequence-specific manner and thus suitable binding domains comprises the following method steps:

a) transforming an expression system comprising

a microorganism having a lethal defect in an essential gene,

a chromosomally integrated insertion element containing a wild-type copy of said gene under the control of a promotor which can be inhibited and permits basal transcription, and a response element,

with a library of expression vectors comprising the deoxyribonucleic acid fragments of the invention and an activating domain,

b) plating out the expression systems on a medium lacking the essential gene product,

c) inhibiting the function of the essential wild-type gene in the insertion element,

d) isolating the growing cell cultures and sequencing the DNA fragment of the invention or the corresponding peptide sequence.

The method is illustrated by the following examples, without being restricted thereto:

Preparation of an Insertion Element Containing a Response Element

A double-stranded DNA sequence containing the wild-type E2F binding site (FIG. 2A), highlighted in gray) in triplicate (3xE2Fwt sequence, Seq. ID No. 7) was prepared as follows:

10 μl each of 10 μM single-stranded oligonucleotides 3xE2F wtsense Seq. ID No. 7 and 3xE2F wtas Seq. ID No. 8 were heated at 95° C. in 100 μl 10 mM Tris-HCl pH 7.9, 50 mM NaCl, 10 mM MgCl₂, 1 mM dithiothreitol for 5 min and then cooled slowly (within 3 h) to room temperature.

Seq. ID No 7

AAAGCGCGCGAAACTAAAGCGCGCGAAACTAAAGCGCGCGAAACTAGCT

Seq. ID No 8

AGTTTCGCGCGCTTTGATTTCGCGCGCTTTGATTTCGCGCGCTTTGATC

The 3xE2Fwt sequence is cloned into the KpnI and SacI restriction cleavage sites of the pGL-2 vector (Promega, Madison, Wis. USA). For this purpose, 3 μg of pGL-2 were digested with in each case 20 u of KpnI and SacI and purified via an agarose gel. The DNA is then removed from remnants of agarose by using the QiaQuick Gel Extraction Kit (Qiagen/Hilden, Germany) according to the manufacturer's instructions and taken up in 50 μl of water. 10 μl of the purified vector were ligated with 1 μl of the double-stranded oligo with the aid of T4 DNA ligase in a 20 μl mixture at 25° C. for 2 h.

For transformation, the E. coli K12 strain “Goldstar” (Stratagene, La Jolla, San Diego, USA) was incubated with shaking at 200 rpm and grown in LB medium containing 100 μg/ml ampicillin at 37° C. overnight. On the next morning, 200 ml of fresh medium were inoculated with 1 ml of the bacterial culture and incubated with shaking at 200 rpm at 37° C. until an optical density of 0.565 at 595 nm was reached. The culture was then cooled to 4° C. and removed by centrifugation at 2500× g. The supernatant was discarded and the pelletted bacteria were taken up in 7.5 ml of LB medium containing 10% (w/v) polyethylene glycol 6000, 5% dimethyl sulfoxide, 10 mM MgSO₄, 10 mM (Promega, Madison, USA), pH 6.8., incubated on ice for one hour, shock-frozen in liquid nitrogen and stored at −80° C. For transformation, 10 μl of the ligation mixture were taken up in 100 μl 100 mM KCl, 30 mM CaCl₂, 50 mM MgCl₂ and incubated on ice with 100 μl of the thawed bacteria for 20 min. After incubating at room temperature for 10 minutes, the bacteria were admixed with 1 ml of LB medium and incubated with shaking at 37° C. for one hour. Subsequently, the mixture was streaked out on LB agar plates containing 100 μg/ml ampicillin and incubated at 37° C. overnight. Individual clones were isolated and grown in 3 ml of LB medium containing 100 μg/ml ampicillin at 37° C. overnight. Plasmid DNA was isolated from the bacteria and purified using the QIAprep Spin Miniprep Kit (Qiagen/Hilden, Germany) according to the manufacturer's instructions. Positive clones were identified by means of PCR as follows: 1 μl of DNA, 0.5 μl, equivalent to 5 U, of Taq polymerase (Promega, Madison, USA), 5 μl of 10× reaction buffer (Promega, Madison, USA), 0.4 μl of deoxynucleotide triphosphate (10 μM each), 4 μl of 12.5 mM MgCl₂, and 0.3 μl each of the 100 μm oligonucleotides and distilled water to 50 μl were heated at 94° C. for 3 min. This was followed by 30 cycles at 94° C. for 20 s, at 50° C. for 20 s and at 72° C. for 45 s, followed by an incubation at 72° C. for 5 min. The PCR products were fractionated by agarose gel electrophoresis, thus determining their size. Bacterial colonies whose plasmid DNA had a PCR product of the expected size were incubated with shaking at 37° C. and 250 rpm in 50 ml of LB medium containing 100 μg/ml ampicillin for 13 h. The plasmid DNA was then isolated from the bacteria and purified with the aid of the Qiagen Midi Prep Kit (Qiagen/Hilden, Germany). The DNA was resuspended in distilled water, followed by isolating the thus amplified 3xE2Fwt sequence from the vector. For this purpose, 3 μg of said plasmid were completely digested with 20 U each of XmaI and MluI for 3 hours. The mixture was then fractionated via an agarose gel and the 3xE2Fwt insertion sequence migrating at 68 bp was cut out using a scalpel. At the same time, 3 μg of pHISi (Clontech, Heidelberg, Germany) were completely digested with 20 U each of XmaI and MluI for 3 hours and purified via an agarose gel. The DNA samples were then removed from agarose by using the QiaQuick Gel Extraction Kit (Qiagen/Hilden, Germany) according to the manufacturer's instructions and taken up in each case in 50 μl of water. For ligation, 3 μl of the pHISi vector digested with XmaI and MluI were incubated with 10 μl of the 3xE2Fwt insertion sequence isolated using XmaI and MluI in a 20 μl mixture together with 1.5 μl of T4 DNA ligase at room temperature for 3 hours. For transformation, the E. coli K12 strain “Goldstar” (Stratagene, La Jolla, San Diego, USA) was incubated with shaking at 200 rpm and grown in LB medium containing 100 μg/ml ampicillin at 37° C. overnight. On the next morning, 200 ml of fresh medium were inoculated with 1 ml of the bacterial culture and incubated with shaking at 200 rpm at 37° C. until an optical density of 0.565 at 595 nm was reached. The culture was then cooled to 4° C. and removed by centrifugation at 2500×g. The supernatant was discarded and the pelletted bacteria were taken up in 7.5 ml of LB medium containing 10% (w/v) polyethylene glycol 6000, 5% dimethyl sulfoxide, 10 mM MgSO₄, 10 mM (Promega, Madison, USA), pH 6.8., incubated on ice for one hour, shock-frozen in liquid nitrogen and stored at −80° C. For transformation, 10 μl of the ligation mixture were taken up in 100 μl 100 mM KCl, 30 mM CaCl₂, 50 mM MgCl₂ and incubated on ice with 100 μl of the thawed bacteria for 20 min. After incubating at room temperature for 10 minutes, the bacteria were admixed with 1 ml of LB medium and incubated with shaking at 37° C. for one hour. Subsequently, the mixture was streaked out on LB agar plates containing 100 μg/ml ampicillin and incubated at 37° C. overnight. Individual clones were isolated and grown in 3 ml of LB medium containing 100 μg/ml ampicillin at 37° C. overnight. Plasmid DNA was isolated from the bacteria and purified using the QIAprep Spin Miniprep Kit (Qiagen/Hilden, Germany) according to the manufacturer's instructions. Positive clones were identified by digestion with XmaI and MluI. The corresponding bacteria were incubated with shaking at 37° C. and 250 rpm in 50 ml of LB medium containing 100 μg/ml ampicillin for 13 h. The plasmid DNA was then isolated from the bacteria and purified with the aid of the Qiagen Midi Prep Kit (Qiagen/Hilden, Germany). The DNA was resuspended in distilled water. The resulting plasmid is referred to as pHISi3xE2Fwt (Seq. ID No. 9).

Preparation of Transformed HIS⁻ Yeast Strains

In plasmid pHISi3xE2Fwt, the HIS3 gene of the vector is put under the control of the insertion element, in addition to the basal HIS3 promotor present in said vector. The yeast strain YM 4271 (Clontech, Heidelberg, Germany) is transformed as follows with the thus prepared pHISi-3xE2Fwt vector according to the manufacturer's instructions (Yeast Protocols Handbook, Clontech, Heidelberg, Germany, pp. 20-21, 1999, Clontech Laboratories Inc.), using 1 μg of the Xho I-linearized plasmid:

1 μg of pHISi3xE2Fwt was completely linearized with 10 U of XhoI for 1 hour, purified using the QiaQuick PCR Purification Kit (Qiagen/Hilden, Germany) according to the manufacturer's instructions and taken up in 50 μl of water. A plurality of colonies of the yeast strain YM 4271 (Clontech, Heidelberg, Germany) were grown in 50 ml of YPD medium up to an optical density at 600 nm of 1.5 at 30° C. and then used to inoculate 300 ml of YPD medium so that the culture had an optical density of 0.2-0.3 at 600 nm. This culture was incubated with shaking at 30° C. until an optical density of 0.4-0.6 at 600 nm was reached. The culture was then removed by centrifugation at 1000× g for 5 minutes, and the cells were taken up in 1.5 ml of 100 mM lithium acetate pH 7.5, 10 mM Tris-HCl pH 7.5, 1 mM EDTA. 100 μl of said cells, 50 μl of the linearized plasmid and 100 μg of heat-denatured salmon sperm DNA were admixed with 600 μl of a PEG/LiAc solution (100 mM lithium acetate pH 7.5, 10 mM Tris-HCl pH 7.5, 1 mM EDTA, 40% (w/v) polyethylene glycol 4000) and incubated with shaking at 30° C. for 30 min. After adding 70 μl of dimethyl sulfoxide, the mixture was incubated at 42° C. for 15 min. The cells were subsequently removed by centrifugation, washed in 1 ml of 10 mM Tris-HCl pH 7.5, 1 mM EDTA and plated out in their entirety on a minimal medium agar plate without addition of histidine, on which 12 colonies had grown after 7 days at 30° C.

The low basal expression of HIS3 permits selection of transformed YM 4271 yeasts which contain the insertion element incorporated in their chromosomal DNA by plating out the cell cultures on SD agar plates containing histidine-free medium. The YM 4271 yeast strains growing under these conditions are isolated.

Selection of a Particularly Suitable Transformed HIS⁻ Yeast Strain

12 colonies resulting from independent recombination events were subsequently streaked out on minimal medium agar plates which contained no histidine but increasing amounts of the HIS3 antagonist 3-aminotriazole (3-AT): 0 mM, 0.5 mM, 1 mM, 1.5 mM, 2 mM, 3 mM, 6 mM, 9 mM, 12 mM, 15 mM, 18 mM, 30 mM, 45 mM; one clone was suppressed markedly already at 0.5 mM and-completely at 2 mM; this clone and another clone did not grow any more at 3 mM, while growth of the remaining 10 clones was inhibited only at >30 mM.

The growth of one of these colonies, referred to as yeast3xE2Fwt was completely suppressed already at a 3-AT concentration of 2 mM (FIG. 2D) without antagonist FIG. 2C)). This colony was isolated and used for the subsequent tests.

The selected strain yeast3xE2Fwt has an advantageous, very low basal HIS3 expression level which is competitively inhibited already by low amounts of 3-AT. The strain is referred to as YM 4271 3xE2Fwt.

Generation of a Library Containing DNA Sequence Specific Binding Partners

3 μg of pGADD424 (Clontech/Heidelberg, Germany, Seq. ID No. 11) were completely digested with 20 u each of EcoRI and BamHI and purified via an agarose gel. 1 μg of the oligonucleotide Seq. ID No. 10 which encodes double-stranded peptide domains was synthesized and digested with 10 u each of EcoRI and BamHI for 3 h and purified via an agarose gel. The DNA was then removed from agarose remnants by using the QiaQuick Gel Extraction Kit (Qiagen/Hilden, Germany) according to the manufacturer's instructions and taken up in 50 μl of water. 3 μl of the vector were ligated with 0.1 μl, 0.3 μl and 1 μl of the oligonucleotide with the aid of T4 DNA ligase in 20 μl mixtures at room temperature for 3 h. The mixtures were subsequently shaken with in each case 200 μl of n-butanol and centrifuged at 13000 rpm in a benchtop centrifuge for 10 minutes. The supernatants were discarded and butanol remnants were removed from the DNA by evaporation under reduced pressure, and said DNA was then taken up in each case in 10 μl of water. For transformation, 500 ml of an exponentially growing bacterial culture of the E. coli K12 strain TOP10 were centrifuged at 4000×g for 10 min, resuspended in 500 ml of cold water and centrifuged again. This procedure was repeated twice. The bacteria were then taken up in 7.5 ml of 10% glycerol (v/v) and shock-frozen in the form of 40 μl aliquots in liquid nitrogen. For the actual transformation, 5 μl each of the above-described purified ligation mixtures were mixed with an aliquot of the bacteria, which had been thawed on ice, and transferred into a cold electroporation cuvette. After an electric pulse of 1.8 kV at 200 Ω and 25 μF, the bacteria were incubated with shaking at 37° C. and 250 rpm in 1 ml of LB medium for one hour and then incubated on LB agar plates containing 100 μg/ml ampicillin at 37° C. overnight. On the next day, the clones of the densely grown plates were scraped off and incubated with shaking at 37° C. and 250 rpm in 250 ml of LB medium containing 100 μg/ml ampicillin for 3 h. The plasmid DNA was then isolated from the bacteria and purified with the aid of the Qiagen Maxi Prep Kit (Qiagen/Hilden, Germany). The DNA was resuspended in distilled water and had a concentration of 1.3 μg/μl.

Seq ID No 10

GGGAATTCGATCCTGCTGCTCTAAAACGTGCTAGANNCACTGAANNNNNN
AGGCGTNNNCGTGCGAGAAAGTTGCAACTTGAAGACAAGGTTGAAGAATT
GCTTTCGAAAAATTATCACTTGGAAAATGAGGTTGCCAGATTAAAGAAAT
TAGTTGGCGAACGCTGAGGATCCCC:

Finding Sequence-Specifically Binding Biomorphic Transcription Factors

The cDNA library generated is transformed into the yeast strain yeast3xE2Fwt according to the manufacturer's instructions (Yeast protocol handbook, Clontech, Heidelberg, Germany) as follows:

A plurality of colonies of the yeast strain YM 4271 3xE2Fwt were grown in 50 ml of YPD medium at 30° C. to an optical density of 1.5 at 600 nm and used for inoculating 300 ml of YPD medium so that the culture had an optical density of 0.2-0.3 at 600 nm. This culture was incubated with shaking at 30° C. until the optical density at 600 nm was 0.4-0.6. The culture was then removed by centrifugation at 1000×g for 5 minutes and the cells were taken up in 1.5 ml of 100 mM lithium acetate pH 7.5, 10 mM Tris-HCl pH 7.5, 10 mM EDTA. 1 ml of cells, 50 μg of the library and 2000 μg of heat-denatured salmon sperm DNA were admixed with 6 ml of a PEG/LiAc solution (100 mM lithium acetate pH 7.5, 10 mM Tris-HCl pH 7.5, 1 mM EDTA, 40% (w/v) polyethylene glycol 4000) and incubated with shaking at 30° C. for 30 min. After adding 700 μl of dimethyl sulfoxide, the mixture was incubated at 42° C. for 15 min. The cells were then removed by centrifugation, washed in 10 mM Tris-HCl pH 7.5, 1 mM EDTA and plated out in their entirety on 6 minimal medium agar plates which contained 2 mM 3-aminotriazole but no histidine and leucine and on which 153 colonies had grown after 7 days at 30° C.

Yeast strains which contain both the insertion element and a component of the library, including the pGAD424-vector Leu2 marker, are selected by being plated out on SD agar plates without histidine and leucine.

In order to select those yeast cells in which the HIS3 expression level has increased due to binding of a biomorphic transcription factor of the library to the trimerized E2F binding site, 2 mM 3-aminotriazole is added to the agar plates.

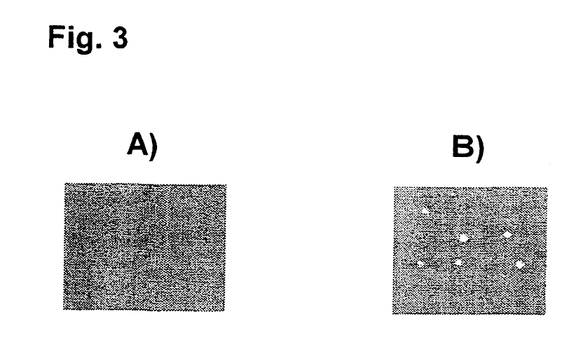

While only one yeast colony grew after control transformation of yeast3xE2Fwt with 50 μg of pGAD424 alone (FIG. 3A)), presumably due to unspecific reverse mutation, transformation with 50 μg of the library produced 153 colonies (FIG. 3B, detail).

The yeast colonies growing under the selection conditions provided have been transformed with library components which code for expressible biomorphic transcription factors whose action is specifically directed toward the HIS3 gene and which contain the E2F binding site, the only multicopy sequence of the HIS3 promotor (HIS3 expression can be regulated only by factors binding to multiple sequence repeats, since only binding of a plurality of transcription factors results in a cooperative and thus steep increase in transcription initiation. Consequently, these colonies contain library members whose expression products bind specifically to the trimerized E2F binding site of the HIS3 gene construct.

In order to determine the sequence of these library members, the plasmids of 32 of said yeast cell cultures were isolated, transformed into E. coli and propagated (Yeast protocols handbook, Clontech, Heidelberg, Germany).

For this purpose, individual yeast colonies were incubated with shaking in 0.5 ml of SD minimal medium without added histidine and leucine at 30° C. overnight. On the next day, the cells were pelletted by centrifugation and the medium was removed so as to leave approximately 50 μl. The cells were resuspended in said remaining medium by vortexing and a spatula tipful of the cell wall-destroying enzyme lyticase was added. The cells were incubated with shaking at 37° C. for one hour and then admixed with 10 μl of a 20% sodium dodecyl sulfate solution. The cells were lysed by 1 min of vortexing, brief freezing at −20° C. and rapid thawing. Cell debris was removed by centrifugation at 14000 rpm and the plasmid DNA present in the supernatant was purified of contaminations by using the QiaQuick PCR Purification Kit and then taken up in 50 μl of water. The plasmid DNA was subsequently transformed into bacteria and propagated in order to obtain larger amounts of said plasmids. For transformation, the E. coli K12 strain “Goldstar” (Stratagene, La Jolla, San Diego, USA) was incubated with shaking at 200 rpm and grown in LB medium containing 100 μg/ml ampicillin at 37° C. overnight. On the next morning, 200 ml of fresh medium were inoculated with 1 ml of the bacterial culture and incubated with shaking at 200 rpm at 37° C. until an optical density of approximately 0.5 at 595 nm was reached. The culture was then cooled to 4° C. and removed by centrifugation at 2500×g. The supernatant was discarded and the pelletted bacteria were taken up in 7.5 ml of LB medium containing 10% (w/v) polyethylene glycol 6000, 5% dimethyl sulfoxide, 10 mM MgSO₄, 10 mM (Promega, Madison, USA), pH 6.8., incubated on ice for one hour, shock-frozen in liquid nitrogen and stored at −80° C. For transformation, 20 μl of the isolated plasmid DNA were taken up in 100 μl 100 mM KCl, 30 mM CaCl₂, 50 mM MgCl₂ and incubated on ice with 100 μl of the thawed bacteria for 20 min. After incubating at room temperature for 10 minutes, the bacteria were admixed with 1 ml of LB medium and incubated with shaking at 37° C. for one hour. Subsequently, the mixture was streaked out on LB agar plates containing 100 μg/ml ampicillin and incubated at 37° C. overnight. A single clone of each transformation was grown in 3 ml of LB medium at 37° C. overnight. The plasmids were subsequently prepared and purified using the Qiagen Tip20 Kit according to the manufacturer's instructions (Qiagen, Hilden, Germany).

The plasmids are sequenced using the oligonucleotide 5′-GATGTATATAACTATCTATTCG-3′ (Seq. ID No. 12) is carried out using standard methods known to the skilled worker (e.g. according to Sanger, using a sequencer).

The following peptide sequences (Table 1, amino acids 1 to 20 shown) were determined as domains which specifically recognize the 3xE2Fwt sequence:

TABLE 1

| S q. ID | |
|---|---|
| No. | 10 20 |
| 13 | D P A A L K R A R G T E V V R R G R A R |
| 14 | D P A A L K R A R C T E V M R R Q R A R |
| 15 | D P A A L K R A R P T E N V R R G R A R |
| 16 | D P A A L K R A R N T E V T R R S R A R |
| 17 | D P A A L K R A R V T E N S R R D R A R |
| 18 | D P A A L K R A R H T E T S R R I R A R |
| 19 | D P A A L K R A R V T E V I R R G R A R |
| 20 | D P A A L K R A R I T E G I R R L R A R |
| 21 | D P A A L K R A R S T E L N R R G R A R |
| 22 | D P A A L K R A R G T E R L R R G R A R |
| 23 | D P A A L K R A R G T E A T R R V R A R |
| 24 | D P A A L K R A R C T E E V R R W R A R |
| 25 | D P A A L K R A R C T E V Q R R G R A R |
| 26 | D P A A L K R A R D T E M L R R C R A R |
| 27 | D P A A L K R A R D T E M V R R A R A R |
| 28 | D P A A L K R A R G T E V V R R C R A R |
| 29 | D P A A L K R A R G T E V V R R C R A R |
| 30 | D P A A L K R A R I T E N A R R G R A R |
| 31 | D P A A L K R A R C T E E M R R G R A R |
| 32 | D P A A L K R A R C T E P S R R G R A R |
| 33 | D P A A L K R A R N T E S G R R T R A R |
| 34 | D P A A L K R A R D T E G D R R R R A R |
| 35 | D P A A L K R A R G T E A E R R G R A R |
| 36 | D P A A L K R A R R T E L L R R H R A R |
| 37 | D P A A L K R A R I T E N A R R G R A R |
| 38 | D P A A L K R A R I T E M G R R K R A R |
| 39 | D P A A L K R A R C T E Y C R R I R A R |
| 40 | D P A A L K R A R G T E E Y R R H R A R |
| 41 | D P A A L K R A R S T E L T R R I R A R |
| 42 | D P A A L K R A R G T E P V R R S R A R |
| 43 | D P A A L K R A R G T E A E R R G R A R |
| 44 | D P A A L K R A R D T E A S R R M R A R |
| 45 | D P A A L K R A R N T E S G R R T R A R |

The domains have at those positions which are crucial for sequence-specific binding amino acids with very similar properties, for example valine and isoleucine (shown with shading). A consensus sequence (Seq. ID No. 13) can be derived from the individual sequences.

The result of comparing the amino acid sequence derived from the DNA sequences of these library members is that in all 4 positions indicated in Seq. ID No. 1 with X amino acid residues with short side chains (G, V, C, S) dominate (Table 2). The frequent appearance of the amino acid glycine in positions X-1 and X-4 is particularly distinctive here.

TABLE 2

| | G | M | P | H | N | C | Q | S | T | Y | A | I | L | F | W | V | D | E | K | R |
|---|---|---|---|---|---|---|---|---|---|---|---|---|---|---|---|---|---|---|---|---|
| Position | 8 | 0 | 1 | 1 | 3 | 6 | 0 | 2 | 0 | 0 | 0 | 4 | 0 | 0 | 0 | 2 | 4 | 0 | 0 | 1 |
| X-1 | | | | | | | | | | | | | | | | | | | | |
| Position | 2 | 3 | 2 | 0 | 4 | 0 | 0 | 2 | 1 | 1 | 4 | 0 | 3 | 0 | 0 | 6 | 0 | 3 | 0 | 1 |
| X-2 | | | | | | | | | | | | | | | | | | | | |
| Position | 3 | 2 | 0 | 0 | 1 | 1 | 1 | 4 | 3 | 1 | 2 | 2 | 3 | 0 | 0 | 6 | 1 | 2 | 0 | 0 |
| X-3 | | | | | | | | | | | | | | | | | | | | |
| Position | 11 | 1 | 0 | 2 | 0 | 3 | 1 | 2 | 2 | 0 | 1 | 3 | 1 | 0 | 1 | 1 | 1 | 0 | 1 | 1 |
| X-4 | | | | | | | | | | | | | | | | | | | | |

It is possible, from comparing the amino acid sequence of the binders, to derive a consensus sequence of the recognition amino acids (Pos. X-1: G or C, Pos. X-2: V, Pos X-3: V, Pos. X-4: G) of the novel peptides, which consensus sequence, however, was not present as separate sequence among the library members analyzed. However, the reason for this may be that, with an estimated efficiency of the library transformation of approx. 200,000 transformation events and with a ratio of vector (with insert) to vector (without insert) of 1:5, only approximately 40,000 different library members were transformed. Therefore, with a library complexity of 104,000 coding members, each member was transformed into the yeast with a probability of 0.38.

1. A biomorphic peptide comprising an amino acid sequence according to Seq. ID No. 1 or having an amino acid sequence which is at least 75% homologous to Seq. ID No. 1, which peptide can specifically bind to a nucleic acid sequences, with the exception of the amino acid sequences having the amino acids N in position 10, A in position 13, A or L in position 14, and S in position 17.

2. The biomorphic peptide as claimed in claim 1, comprising an amino acid sequence according to Seq. ID No. 1 or with an amino acid sequence which is at least 75% homologous to Seq. ID No. 1, which peptide can bind specifically to a nucleic acid sequences, with the amino acid X in position 10 being A, B, C, D, E, F, G, H I, K, L, M, P, Q, R, S, T, V, W, Y, Z,
the amino acid in position 13 being B, C, D, E, F, G, H I, K, L, M, N, P, Q, R, S, T, V, W, Y, Z, the amino acid in position 14 being B, C, D, E, F, G, H I, K, M, N, P, Q, R, S, T, V, W, Y, Z and the amino acid in position 17 being A, B, C, D, E, F, G, H I, K, L, M, N, P, Q, R, T, V, W, Y, Z.

3. The biomorphic peptide as claimed in either of claims 1 and 2, which can specifically bind a nucleic acid sequences, obtainable by
a) transforming an expression system comprising
a microorganism having a lethal defect in an essential gene,
a chromosomally integrated insertion element containing a wild-type copy of said gene under the control of a promotor which can be inhibited and permits basal transcription, and a response element,
with a library of expression vectors comprising the deoxyribonucleic acid fragments comprising nucleic acid sequences according to Seq. ID No. 3 or to a structural variant thereof, which encode biomorphic peptides as claimed in either of claims 1 and 2, and an activating domain,
b) plating out the expression systems on a medium lacking the essential gene product,
c) inhibiting the basal transcription of the essential wild-type gene in the insertion element,
d) isolating the growing cell cultures and sequencing the DNA fragment of the invention or the corresponding peptide sequence.

4. The biomorphic peptide as claimed in any of claims 1 to 3, characterized in that said peptide is present in a homo- and/or heterodimeric form.

5. The biomorphic peptide as claimed in any of the preceding claims, characterized in that at least two peptides selected from the group according to Seq. ID No. 1 or having an amino acid sequence which is at least 75% homologous to Seq. ID No. 1 are linked via a linker.

6. The biomorphic peptide as claimed in claim 5, characterized in that it contains complexing agents or leucine zippers as linkers.

7. The biomorphic peptide as claimed in any of the preceding claims, comprising an amino acid sequence according to Seq. ID No. 2.

8. The biomorphic peptide as claimed in any of the preceding claims, comprising an amino acid sequence according to Seq. ID No. 1, an amino acid sequence which is at least 75% homologous to Seq. ID No. 1 or to Seq. ID No. 2 and nuclear transport signal domains, transcription-activating domains or transcription-inhibiting domains.

9. The use of biomorphic peptides comprising an amino acid sequence according to Seq. ID No. 1, an amino acid sequence which is at least 75% homologous to Seq. ID No. 1 or to Seq. ID No. 2 for producing pharmaceuticals for the treatment of gene regulation disorders.

10. A nucleic acid comprising nucleic acid sequences according to Seq. ID No. 3 or a nucleic acid sequence coding for an amino acid sequence which is at least 75% homologous to Seq. ID No. 1, which encode biomorphic peptides as claimed in claims 1 to 8.

11. The nucleic acid as claimed in claim 10, characterized in that it comprises a leucine-zipper sequence.

12. The nucleic acid as claimed in claim 10 or 11, characterized in that it comprises regions encoding nuclear transport signal domains, transcription-activating domains or transcription-inhibiting domains.

13. A vector comprising nucleic acids as claimed in claim 10 or 11.

14. The nucleic acid as claimed in claims 10 to 12, characterized in that said nucleic acid is a DNA or cDNA.

15. The use of nucleic acids or vectors as claimed in claims 10 to 13 for producing gene therapy pharmaceuticals for the treatment of gene regulation disorders.

16. A library comprising deoxyribonucleic acids which comprise a nucleic acid sequence according to Seq. ID No. 3 or a nucleic acid sequence coding for an amino acid sequence which is at least 75% homologous to Seq. ID No. 1 and a region encoding a transcription-activating domain.

17. The library as claimed in claim 16, characterized in that the deoxyribonucleic acids additionally comprise a region encoding a nuclear transport signal domain and/or a leucine zipper.

18. A peptide library comprising biomorphic transcription factors which comprise amino acid sequences as claimed in any of claims 1 to 8.

19. A method for finding biomorphic transcription factors which bind DNA sequence-specifically, which comprises the following method steps:
a) transforming an expression system comprising
a microorganism having a lethal defect in an essential gene,
a chromosomally integrated insertion element containing a wild-type copy of said gene under the control of a promotor which can be inhibited and permits basal transcription, and a response element,
with a library of expression vectors comprising the deoxyribonucleic acid fragments as claimed in any of claims 10 to 12 and an activating domain,
b) plating out the expression systems on a medium lacking the essential gene product,
c) inhibiting the basal transcription of the essential wild-type gene in the insertion element,
d) isolating the growing cell cultures and sequencing the DNA fragment of the invention or the corresponding peptide sequence.

20. A biomorphic peptide comprising an amino acid sequence according to Seq. ID No. 1 or to a structural variant, characterized in that the amino acid at position X-1 is G, C, I, D, N, S, P, H or R, the amino acid at position X-2 is V, A, N, M, L, E, G, P, S, T, Y or R, the amino acid at position X-3 is V, S, G, T, L, M, A, I, E, N, C, Q, Y or D, the amino acid at position X-4 is G, C, I, H, S, T, M, Q, A, L, W, V, D, K or R.

21. The biomorphic peptide as claimed in claim 20, characterized in that the amino acid at position X-1 is G, C, I, or D, the amino acid at position X-2 is V, A, or N, the amino acid at position X-3 is V or S, the amino acid at position X-4 is G.

22. The biomorphic peptide as claimed in claim 20 or 21, characterized in that the amino acid at position X-1 is G, the amino acid at position X-2 is V, the amino acid at position X-3 is V, the amino acid at position X-4 is G.

23. An artificial biomorphic factor comprising a peptide with a sequence as claimed in claims 20 to 22.

24. An artificial biomorphic transcription factor or repressor as claimed in claim 23, which specifically binds the E2Fwt sequence.
